(19) United States
(12) Patent Application Publication
(10) Pub. No.: US 2020/0272276 A1
(43) Pub. Date: Aug. 27, 2020

(54) Embedded Vital Sign Monitoring in Display Screens

(71) Applicant: OSRAM Opto Semiconductors GmbH, Regensburg (DE)

(72) Inventors: Christoph Goeltner, Cupertino, CA (US); Tim Böscke, Regensburg (DE); Naresh Shetty, Pleasanton, CA (US)

(21) Appl. No.: 16/457,825

(22) Filed: Jun. 28, 2019

Related U.S. Application Data
(60) Provisional application No. 62/808,886, filed on Feb. 22, 2019.

(51) Int. Cl. G06F 3/042; A61B 5/00

(52) U.S. Cl. CPC G06F 3/0421; A61B 5/0059; A61B 5/6826

(57) ABSTRACT

A mobile computing device is disclosed. In an embodiment a mobile computing device includes a display screen including a top surface and a bottom surface, and a vital sign monitoring (VSM) sensor located within the display screen or beneath the bottom surface of the display screen, wherein the VSM sensor is configured to measure one or more vital sign parameters of a user that places a body part on the top surface of the display screen above the VSM sensor.

### CROSS-REFERENCE TO RELATED APPLICATIONS

This application claims the priority of U.S. Patent Application No. 62/808,886 filed on Feb. 22, 2019, which application is hereby incorporated herein by reference.

### TECHNICAL FIELD

This disclosure relates to vital sign monitoring, and specifically to systems and methods for embedding vital sign monitoring sensors within computing device display screens.

### BACKGROUND

Organic light-emitting diodes (OLEDs) are frequently used to make high definition display screens for televisions, computers, and mobile computing devices such as smart phones. In particular, the relative thinness of OLED screens is attractive for mobile computing devices due to the size limitations of such devices and the desire achieve a thin, sleek profile or design aesthetic.

Traditionally, buttons or sensors are integrated into mobile computing devices through bezels or other openings or cut-outs in the screen and/or body of the mobile computing device. However, manufacturers prefer to minimize the number of bezels and openings in a mobile computing device to simplify manufacturing and improve design aesthetic. Thus there exists a need for ways to integrate sensors and other extraneous components to a mobile computing device in a manner that reduces manufacturing complexity while increasing design aesthetic.

### SUMMARY

Embodiments provide a system and a method including an embedded vital sign monitoring (VSM) sensor within a display screen of a mobile computing device. The vital sign monitoring sensor may be located within the display screen or beneath the display screen. In one implementation, the vital sign monitoring sensor may include a detector. Light emitted by the display screen may reflect off a person's finger that is touching the screen above the sensor. The sensor detects the reflected light, and the information may be used to determine various vital sign parameters of the person. In another implementation, the vital sign sensor beneath the display screen may include both an emitter and a detector. The emitter may emit light of certain wavelengths, which travel through the display screen and reflect off a person's finger. The reflected light is captured by the detector, and the information is analyzed.

### BRIEF DESCRIPTION OF THE DRAWINGS

FIG. 1 is a block diagram of a VSM sensor embedded in a display screen in accordance with various embodiments;

FIG. 2 is a block diagram of a mobile computing device with an embedded VSM sensor in accordance with various embodiments;

FIG. 3 is a block diagram of a VSM sensor in a display screen with light guides in accordance with various embodiments;

FIG. 4 is a block diagram of another VSM sensor embedded in a display screen in accordance with various embodiments; and

FIG. 5 is a graph of light absorption versus wavelength for a variety of materials within a human body.

These and other features of the present embodiments will be understood better by reading the following detailed description, taken together with the figures herein described. The accompanying drawings are not intended to be drawn to scale. For purposes of clarity, not every component may be labeled in every drawing.

### DETAILED DESCRIPTION OF ILLUSTRATIVE EMBODIMENTS

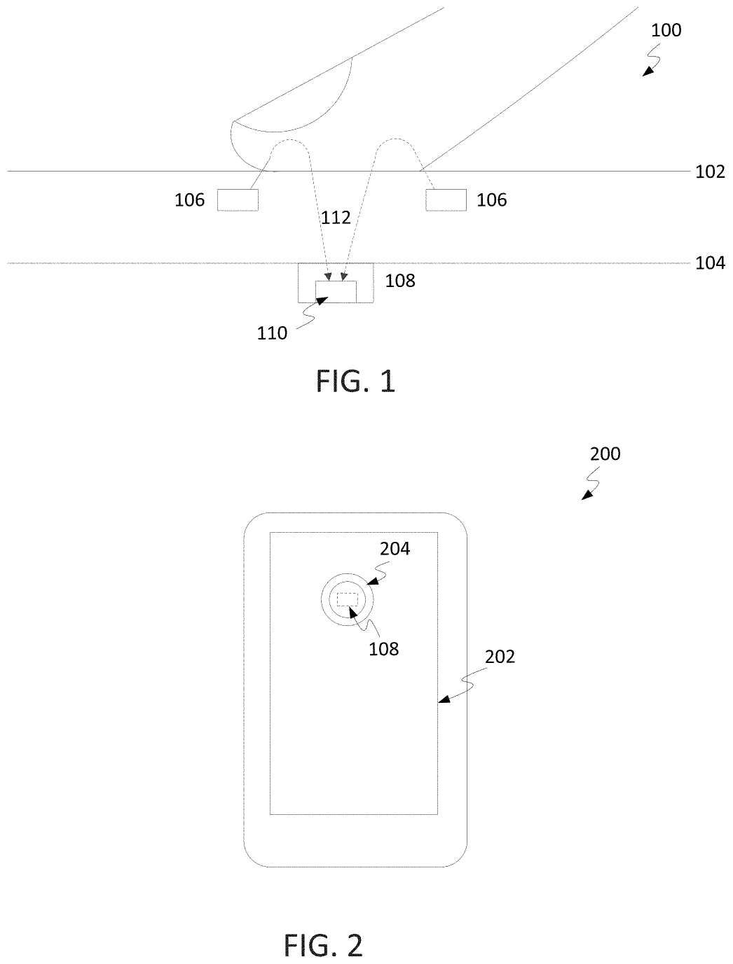

FIG. 1 is a block diagram illustrating a VSM sensor embedded in a display screen 100 in accordance with various embodiments. The display screen 100 includes a top surface 102 that faces the user and a bottom surface 104 that faces the interior of a device that includes the display screen 100. The device may be, for example, a smart phone, a wearable computing device, or other mobile computing device. The display screen 100 may be, for example, an OLED display screen. The display screen 100 may include light emitting components 106 that emit light out through the top surface 102 of the display screen 100. The light emitting components 106 may be, for example, a layer of a light emitting organic compound in the display screen 100.

A VSM sensor 108 may be embedded into the display screen 100. The VSM sensor 108 may include a detector no for detecting various properties of light (e.g., intensity, wavelength). The VSM sensor 108 may be located below the bottom surface 104 of the display screen 100. In alternate implementations, the VSM sensor 108 may be embedded in the semiconductor stack of the display screen (e.g., within the OLED stack). The VSM sensor 108 may be attached the display screen by semiconductor packaging methods known in the art. The VSM sensor 108 may include circuitry for driving the detector no and for signal processing of information obtained by the detector no to calculate various vital sign parameters. For example, the VSM sensor 108 may utilize photoplesythmogrophy (PPG) to calculate the vital sign parameters.

The device may be executing an application that includes functionality for monitoring a user's vital signs. The application may request that the user place their finger on the top surface 102 of the display screen 100, above the VSM sensor 108. Light emitting components 106 in the vicinity of the VSM sensor 108 may emit light beams 112 of certain wavelengths and/or intensities towards the top surface 102. The intensity and wavelength of the light beams 112 may be chosen based on their usefulness in vital sign measurements, and also to provide sufficient brightness for the detector no to detect reflected light. The light beams 112 exit the top surface 102 and into the user's finger, where they penetrate certain distances into the finger and are reflected back out into the display screen 100. The reflected light beams 112 may be detected by the detector no in the VSM sensor 108. The data collected by the VSM sensor 108 may be used to determine various vital sign parameters of the user, such as heart rate or blood oxygen saturation (SpO2).

FIG. 2 illustrates a block diagram of a mobile computing device 200 with an embedded VSM sensor in accordance with various embodiments. The mobile computing device 200 may include a display screen 202, which may be similar to display screen 100 in FIG. 1. The VSM sensor 108 may be located beneath a portion of the display screen 202. When the mobile computing device 200 is executing an application that measures a user's vital signs, the display screen 202 may emit a light pattern 204 in the vicinity of the VSM sensor 108. This indicates that the user should place their finger on top of the light pattern 204 in order for the VSM sensor 108 to take measurements. While light pattern 204 is illustrated as a ring in FIG. 2, in general light pattern 204 may take a variety of shapes or patterns. For example, the light pattern 204 may be chosen to maximize the amount of reflected light detected by the VSM sensor 108.

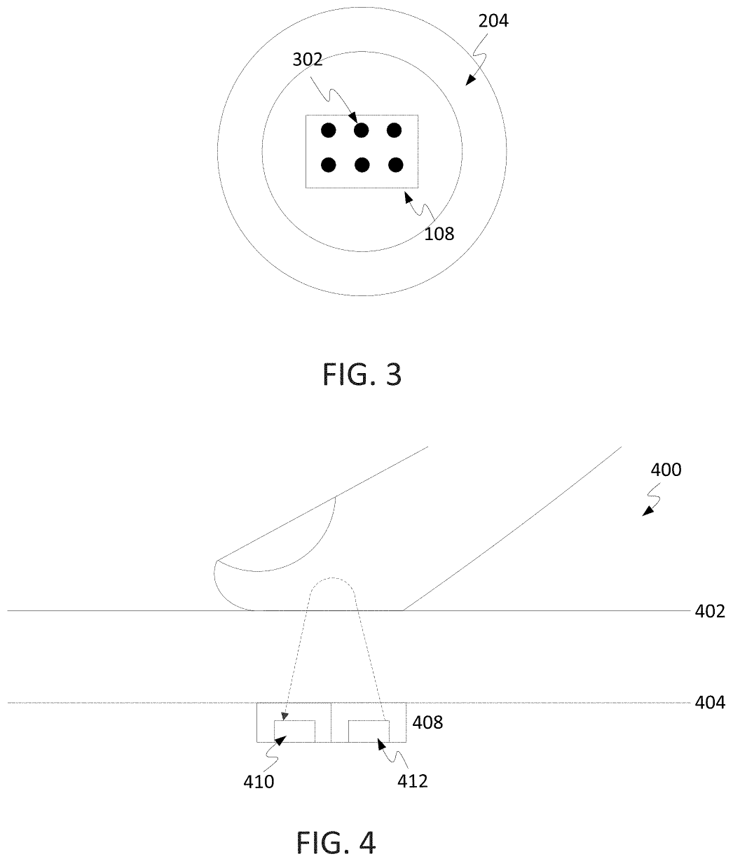

If the reflected light from the light pattern 204 has too low of an intensity when it reaches the VSM sensor 108, light guides may be incorporated into the display screen to increase the intensity of reflected light reaching the VSM sensor 108. This is illustrated in FIG. 3, which shows a number of light guides 302 in the display screen above the VSM sensor 108. The light guides 302 may be, for example, small holes that go from the top surface of the display screen to the bottom surface. The light guides 302 may be sized such that they do not interfere with the pitch of the screen but are still able to guide the reflected light to the VSM sensor 108. Methods for creating holes or other implementations of the light guides 302 in semiconductor stacks are known in the art.

FIG. 4 illustrates another implementation of a VSM sensor embedded in a display screen 400 in accordance with various embodiments. The display screen 400 includes a top surface 402 that faces the user and a bottom surface 404 that faces the interior of a device that includes the display screen 400. The device may be, for example, a smart phone, a wearable computing device, or other mobile computing device. The display screen 400 may be, for example, an OLED display screen.

A VSM sensor 408 may be embedded into the display screen 400. The VSM sensor 408 may include an emitter 412 for emitting light into the display screen 400, and a detector 410 for detecting various properties of light (e.g., intensity, wavelength). The VSM sensor 408 may be located below the bottom surface 404 of the display screen 400. In alternate implementations, the VSM sensor 408 may be embedded in the semiconductor stack of the display screen (e.g., within the OLED stack). The VSM sensor 408 may be attached the display screen by semiconductor packaging methods known in the art. The VSM sensor 408 may include circuitry for driving the detector 410 and emitter 412 and for signal processing of information obtained by the detector 410 to calculate various vital sign parameters. For example, the VSM sensor 408 may utilize photoplesythmogrophy (PPG) to calculate the vital sign parameters.

The device may be executing an application that includes functionality for monitoring a user's vital signs. The application may request that the user place their finger on the top surface 402 of the display screen 400, above the VSM sensor 408. The emitter 412 may emit light beams of certain wavelengths and/or intensities towards the top surface 402. The light beams exit the top surface 402 and into the user's finger, where they penetrate certain distances into the finger and are reflected back out into the display screen 400. The reflected light beams may be detected by the detector 410 in the VSM sensor 408. The data collected by the VSM sensor 408 may be used to determine various vital sign parameters of the user, such as heart rate or blood oxygen saturation (SpO2).

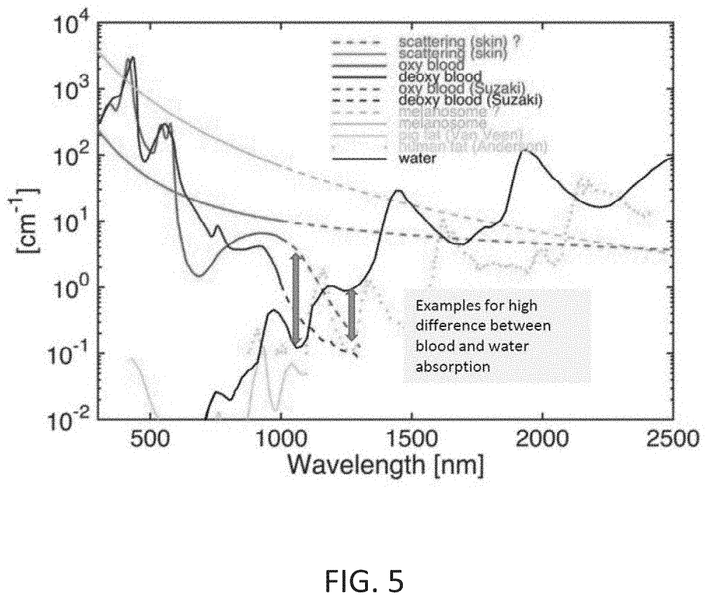

Selection of the appropriate wavelength of light for emitter 412 is important. One challenge is that there may be a significant loss of light intensity when the light path travels from the emitter 412 through the display screen 400, is reflected off a user's finger, and then transmitted back through the display screen 400 to the detector 410. One factor to consider is the effect of light on display screen integrity. For example, wavelengths larger than 1 micrometer are known to go through display screens, such as OLED display screens, without causing harmful degradation to the OLED transistor stack. Another factor to consider is that the wavelength should be chosen such that the detector 410 can detect a sufficient amount of signal of the heart beat induced pressure wave. From a vital signal monitoring point of view, a wavelength should be chosen that shows different absorption for blood than for water. For example, FIG. 5 illustrates that there is a significant difference between blood and water absorption of light at wavelengths of 1200 nm or 1500 nm. If the absorption for blood and water is the same, the water content will make it harder to detect the heart beat signal.

The methods and systems described herein are not limited to a particular hardware or software configuration, and may find applicability in many computing or processing environments. The methods and systems may be implemented in hardware or software, or a combination of hardware and software. The methods and systems may be implemented in one or more computer programs, where a computer program may be understood to include one or more processor executable instructions. The computer program(s) may execute on one or more programmable processors, and may be stored on one or more storage medium readable by the processor (including volatile and non-volatile memory and/or storage elements), one or more input devices, and/or one or more output devices. The processor thus may access one or more input devices to obtain input data, and may access one or more output devices to communicate output data. The input and/or output devices may include one or more of the following: Random Access Memory (RAM), distributed and virtual data storage technologies, floppy drive, CD, DVD, Blu-Ray, magnetic disk, internal hard drive, external hard drive, memory stick, flash drive, solid state memory device, or other storage device capable of being accessed by a processor as provided herein, where such aforementioned examples are not exhaustive, and are for illustration and not limitation.

The computer program(s) may be implemented using one or more high level procedural or object-oriented programming languages to communicate with a computer system; however, the program(s) may be implemented in assembly or machine language, if desired. The language may be compiled or interpreted.

As provided herein, the processor(s) may thus be embedded in one or more devices that may be operated independently or together in a networked environment, where the network may include, for example, a Local Area Network (LAN), wide area network (WAN), and/or may include an intranet and/or the internet and/or another network. The network(s) may be wired or wireless or a combination thereof and may use one or more communications protocols to facilitate communications between the different processors. The processors may be configured for distributed processing and may utilize, in some embodiments, a client-server model as needed. Accordingly, the methods and systems may utilize multiple processors and/or processor devices, and the processor instructions may be divided amongst such single- or multiple-processor/devices.

The device(s) or computer systems that integrate with the processor(s) may include, for example, a personal computer(s), workstation(s), handheld device(s) such as cellular telephone(s) or smartphone(s) or tablet(s), laptop(s), laptop/tablet hybrid(s), handheld computer(s), smart watch(es), or any another device(s) capable of being integrated with a processor(s) that may operate as provided herein. Accordingly, the devices provided herein are not exhaustive and are provided for illustration and not limitation.

References to “a microprocessor” and “a processor”, or “the microprocessor” and “the processor,” may be understood to include one or more microprocessors that may communicate in a stand-alone and/or a distributed environment(s), and may thus be configured to communicate via wired or wireless communications with other processors, where such one or more processor may be configured to operate on one or more processor-controlled devices that may be similar or different devices. Use of such “microprocessor” or “processor” terminology may thus also be understood to include a central processing unit, an arithmetic logic unit, an application-specific integrated circuit (IC), and/or a task engine, with such examples provided for illustration and not limitation.

Furthermore, references to memory, unless otherwise specified, may include one or more processor-readable and accessible memory elements and/or components that may be internal to the processor-controlled device, external to the processor-controlled device, and/or may be accessed via a wired or wireless network using a variety of communications protocols, and unless otherwise specified, may be arranged to include a combination of external and internal memory devices, where such memory may be contiguous and/or partitioned based on the application. Accordingly, references to a database may be understood to include one or more memory associations, where such references may include commercially available database products (e.g., SQL, Informix, Oracle) and also proprietary databases, and may also include other structures for associating memory such as links, queues, graphs, trees, with such structures provided for illustration and not limitation.

References to a network, unless provided otherwise, may include one or more intranets and/or the internet. References herein to microprocessor instructions or microprocessor-executable instructions, in accordance with the above, may be understood to include programmable hardware.

Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems.

Throughout the entirety of the present disclosure, use of the articles “a” and/or “an” and/or “the” to modify a noun may be understood to be used for convenience and to include one, or more than one, of the modified noun, unless otherwise specifically stated. The terms “comprising”, “including” and “having” are intended to be inclusive and mean that there may be additional elements other than the listed elements.

The foregoing description of the embodiments of the present disclosure has been presented for the purposes of illustration and description. It is not intended to be exhaustive or to limit the present disclosure to the precise form disclosed. Many modifications and variations are possible in light of this disclosure. It is intended that the scope of the present disclosure be limited not by this detailed description, but rather by the claims appended hereto.

What is claimed is:

1. A mobile computing device comprising:
a display screen comprising a top surface and a bottom surface; and
a vital sign monitoring (VSM) sensor located within the display screen or beneath the bottom surface of the display screen,
wherein the VSM sensor is configured to measure one or more vital sign parameters of a user that places a body part on the top surface of the display screen above the VSM sensor.

2. The mobile computing device of claim 1,
wherein the display screen is configured to emit light toward the body part, and
wherein the VSM sensor comprises a detector configured to detect light reflected from the body part.

3. The mobile computing device of claim 2, wherein the display screen further comprises one or more light guides above the VSM sensor.

4. The mobile computing device of claim 2, wherein the display screen comprises a layer of a light emitting organic compound configured to emit the light toward the body part.

5. The mobile computing device of claim 1, wherein the VSM sensor comprises:
an emitter configured to emit light toward the body part; and
a detector configured to detect light reflected from the body part.

6. The mobile computing device of claim 5, wherein the emitter is configured to emit light having wavelengths larger than 1 micrometer.

7. The mobile computing device of claim 5, wherein the emitter is configured to emit the light comprising a wavelength having a different absorption for blood than for water.

8. The mobile computing device of claim 5, wherein the VSM sensor is located beneath the bottom surface of the display screen.

9. The mobile computing device of claim 1, wherein the display screen comprises an organic light-emitting diode (OLED) display screen.

10. The mobile computing device of claim 1, wherein the mobile computing device is configured to request that the user places the body part on the top surface of the display screen above the VSM sensor.

11. The mobile computing device of claim 1, wherein the display screen is configured to emit a light pattern in a vicinity of the VSM sensor to request that the user places the body part on top of the light pattern.
